(19) United States
(12) Patent Application Publication
(10) Pub. No.: US 2006/0134608 A1
(43) Pub. Date: Jun. 22, 2006

(54) Chemically amplified electrochemical detection of affinity reaction

(76) Inventors: Lianghong Guo, Beijing (CN); Yuanguang Li, Beijing (CN); Na Wang, Beijing (JP); Fuquan Wang, Beijing (CN); Xiqiang Yang, Beijing (CN); Jing Cheng, Beijing (CN)

Correspondence Address: MORRISON & FOERSTER LLP, 12531 HIGH BLUFF DRIVE, SUITE 100, SAN DIEGO, CA, 92130-2040, US

(21) Appl. No.: 10/537,378

(22) PCT Filed: May 6, 2003
(86) PCT No.: PCT/CN03/00329

§ 371 Date: Jan. 18, 2006

(30) Foreign Application Priority Data
Dec. 3, 2002 (CN) 02153665.1

(51) Int. Cl. C12Q 1/70; C12Q 1/68; G01N 33/567

(52) U.S. Cl. 435/5; 435/6; 435/7.2; 205/777

(57) ABSTRACT

This invention relates generally to the field of electrochemistry. In particular, the invention provides a method for assaying an analyte, using, inter alia, a reactant capable of binding and/or reacting with an analyte to be analyzed on an oxide electrode and a reducing agent not capable of being oxidized directly by said electrode to generate reduced electrochemically active molecule that participates in oxidation-reduction reactions repeatedly to generate an amplified electrochemical signal to determine presence and/or amount of said analyte in said sample.

### TECHNICAL FIELD

This invention relates generally to the field of electrochemistry. In particular, the invention provides a method and a kit for assaying an analyte, using, inter alia, a reactant capable of binding and/or reacting with an analyte to be analyzed on an oxide electrode and a reducing agent not capable of being oxidized directly by said electrode to generate reduced electrochemically active molecule that participates in oxidation-reduction reactions repeatedly to generate an amplified electrochemical signal to determine presence and/or amount of said analyte in said sample.

### BACKGROUND ART

Effort has been made constantly to improve currently available analytical methods, and to develop new methods with the goal to get higher sensitivity, lower cost, and more reproducibility. Typically, for affinity-based biological detection, a label (signal-generating molecule) is attached to a biological molecule, which binds to its complementary partner through a unique biological recognition. The recognition reactions include DNA-DNA, DNA-RNA, antigen-antibody, ligand-receptor, etc. The binding reaction is detected, and sometimes quantified, by measuring the signal emitted from the label in the form of light, current, mass, sound, etc.

In the early days of biological detection, radio-isotopes were employed as labels. They provided adequate sensitivity, but had short shelf life and were hazardous to human health. They were subsequently replaced with enzyme labels and absorption-based detection instrument (e.g., ELISA). The enzyme label is safe but not stable enough for long-term storage. And sensitivity also suffered. Next emerged are fluorescent organic and inorganic molecules, which are both safe and stable. Although they provide higher sensitivity than ELISA, they are still not comparable to radio-isotopes. With complex and expensive laser excitation source and detection optics, instrument cost is also a major disadvantage. Recently, chemiluminescence and electrochemiluminescence are becoming the detection method of choice in clinical diagnostic laboratories, thanks to their ultra-high sensitivity (due to very low background) and stable reagents. As the method still employs optical detection, however, instrument cost remains relatively high.

In parallel with optical detection, electrochemistry has also been employed in chemical and biological analysis along the way. Because of its low instrument cost and simplicity, electrochemical detection has been quite successful in areas where cost and portability are major issues. Examples include ion-selective electrodes, handheld glucose meters and other blood analyzers. However, electrochemical detection of affinity reactions, such as those between antibody and antigen, are not as successful. This is mainly due to the fact that in regular electrochemical detection, labels for the affinity reaction donate or accept only one electron, which is the detected signal. This fact, in combination with the background current from the double-layer charging, severely limits its sensitivity.

Chemical amplification was proposed previously as a way of enhancing electrochemical signal. To implement the strategy, a chemical agent is added to the electrolyte. The detected signal is still electrochemical current of the labeling molecule of the affinity reaction. In the reaction sequence, first the label is oxidized by the electrode. This is followed by a redox chemical reaction between the oxidized label and chemical agent in solution, which reduces the label back to its original redox state. The regenerated label can participate in the first step again. The overall effect is the cycling of the same labeling molecule and repeated electrochemical reaction. This results in signal amplification because more than one electron is extracted from same label.
(1) Electrode reaction: Label (red)-→Label (ox)
(2) Chemical regeneration: Label (ox)+reducing agent-→Label (red)
(3) Repeat (1)

Although chemical amplification was proposed years ago, there has not been much success. The main reason is that, generally speaking, the chemical agent used for the amplification can get oxidized directly at the electrode, giving rise to high background current. The challenge is therefore to maximize amplification effect and at the same time minimize background from the chemical agent.

Affinity reaction is typically detected with the help of a signal-generating molecule, called label, which is attached to one of the affinity pair. As described above, a variety of molecules that generate color, fluorescence, chemiluminescence and electrochemiluminescence, have been employed as labels.

In theory, many biological molecules can be detected directly by electrochemical methods without the use of a label, since some of their components are redox active. For instance, in DNA, guanine has a redox potential of 1.3 V vs. NHE, which is easily accessible on many electrodes. Other bases have higher redox potentials. The sugar moiety of a DNA is also oxidizable. In proteins, the redox potential of tyrosine is 0.82-0.95 V, tryptophan 0.82-1.07 V, and histidine 1.32-1.62 V. However, the kinetics of these oxidation reactions is so slow that they are not practical for detection.

In U.S. Pat. No. 5,871,918, Thorp et al described a method of DNA analysis by indirect electrochemical oxidation of guanine base. In the method, target DNA hybridizes with a probe immobilized on an electrode surface. Transition metal complexes such as ruthenium tris-bipyridine are dissolved in solution, and are used to mediate the electrochemical oxidation of guanine bases in target DNA. The complex is first electrochemically oxidized. It is then reduced back by chemical reaction with guanine. The reduced complex can be oxidized again at the electrode. The drawback of the approach is that the complex produces oxidation current in the absence of any DNA, thus background signal. In addition, because the amount of guanine bases in DNA is small, amplification efficiency is not optimal. In U.S. Pat. No. 6,346,387, the same authors proposed a similar approach for protein analysis.

As mentioned above, electrochemical labels can be used for the analysis of affinity reactions. In U.S. Pat. No. 5,312,527, Mikkelsen et al labeled DNA duplex with a cobalt tris-bipirydine by non-covalent binding and subsequently performed electrochemical detection. DNA duplex is hybridized on a glassy carbon electrode. The cobalt complex is added to the electrolyte in small amount. It binds to the duplex by intercalation, but not to the single-strand. In the absence of target DNA, the complex is uniformly distributed in solution at low concentration, producing some background current. When target DNA is introduced and hybridized with the probe immobilized on the electrode, the complex intercalates into the duplex. The accumulation of the complex on the electrode surface produces current much higher than the complex in solution. This serves as an indication of duplex formation. This approach has the same drawback as Thorp's in that unbound metal complex produces high background current.

Following the conventional way of labeling DNA, Meade et al disclosed in U.S. Pat. No. 6,277,576 a synthetic method of covalently attaching an electrochemical label to the sugar moiety of DNA. Ferrocene, a classical electrochemical compound with fast electrode kinetics, was employed as the label. DNA probe was immobilized on a gold electrode through an electronically conducting thiol molecule. The other area of the surface was covered with an insulating layer. After labeled DNA hybridized with the probe, electrochemical current of ferrocene was detected, while the insulating layer kept the background current at minimum. It is worth pointing out here that there is no amplification mechanism in the disclosed methods.

Enzymes have been used in many occasions as labels for DNA and immunological reactions. Because one enzyme molecule is capable of catalyzing thousands or more of substrates, there is an inherent amplification mechanism. Examples include those disclosed in William Heineman, et al. Anal. Chem. 1996, 68, 2453; I. Willner et al. Anal. Chem, 1996, 68, 3151; and Adam Heller, J. Am. Chem. Soc., 1999, 121, 769. The large size and instability of enzymes have limited their application.

U.S. Pat. No. 6,221,586 employs an approach of using an electrochemically active DNA intercalator as the label, and dissolved ferricyanide as the reducing agent to regenerate oxidized intercalator. Because it is difficult to suppress ferricyanide current on the gold electrode being used, sensitivity is a major problem.

There exists a need in the art for a sensitive and cost-effection assay detection. This invention address this and other related needs in the art.

### DISCLOSURE OF THE INVENTION

Electrode materials and reagents are used for the detection of chemical and biological affinity reactions by the method of chemically amplified electrochemistry. The benefits of the present methods and kits include significantly lower instrument cost than the currently popular fluorescence method, but with a comparable sensitivity.

In one aspect, the present invention is directed to a method for assaying an analyte, which method comprises: a) providing a reactant capable of binding and/or reacting with an analyte to be analyzed on an oxide electrode; b) contacting a sample suspected of containing said analyte with said reactant provided in step a) under suitable conditions to allow binding of said analyte, if present in said sample, to said reactant, wherein said reactant, said analyte, or additional reactant or additional analyte or analyte analog is covalently linked to an electrochemically active molecule in a reduced form, and said contacting brings said electrochemically active molecule into close proximity to said electrode to allow oxidation of said electrochemically active molecule by said electrode; c) allowing reduction of said oxidized electrochemically active molecule back to said reduced form by a reducing agent, wherein said reducing agent is not capable of being oxidized directly by said electrode, and said reduced electrochemically active molecule participates in said oxidation-reduction reactions of steps b) and c) repeatedly to generate an amplified electrochemical signal; and d) assessing said amplified electrochemical signal to determine presence and/or amount of said analyte in said sample.

In another aspect, the present invention is directed to a kit for assaying an analyte, which kit comprises: a) a reactant capable of binding and/or reacting with an analyte to be analyzed on an oxide electrode; b) an additional reactant, analyte, or analyte analog that is covalently linked to an electrochemically active molecule in a reduced form, wherein contacting of said analyte with said reactant on said electrode in the presence of said additional reactant, analyte, or analyte analog that is covalently linked to said electrochemically active molecule brings said electrochemically active molecule into close proximity to said electrode to allow oxidation of said electrochemically active molecule by said electrode; c) a reducing agent, wherein said reducing agent is not capable of being oxidized directly by said electrode, and said reducing agent reduces said oxidized electrochemically active molecule back to said reduced form to participate in repeated oxidation-reduction reactions to generate an amplified electrochemical signal; and d) means for assessing said amplified electrochemical signal to determine presence and/or amount of said analyte in said sample.

### BRIEF DESCRIPTION OF THE DRAWINGS

FIG. 1 illustrates an oxolate amplified current for ruthenium tris(2,2′-bipyridine).

FIG. 2 illustrates cyclic voltammograms of proline with various concentrations of ruthenium tris(2,2′-bipyridine).

FIG. 3 illustrates relationship between amplified current and ruthenium tris(2,2′-bipyridine) concentration.

FIG. 4 illustrates proline amplified current of ruthenium tris(2,2′-bipyridine) at various concentrations.

FIG. 5 illustrates biotin-avidin binding detected by chemically amplified electrochemistry.

### MODES OF CARRYING OUT THE INVENTION

For clarity of disclosure, and not by way of limitation, the detailed description of the invention is divided into the subsections that follow.

A. Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “electrode” refers to an electric conductor or semiconductor through which an electric current enters or leaves a medium. The medium can be an electrolytic solution, a solid, molten mass, gas or vacuum.

As used herein, “oxide electrode” refers to an electric conductor or semiconductor composed of a metal oxide or a non-metal oxide. The oxide may exist as a stable state, and is therefore called “native oxide”. Alternatively, the oxide may be generated only after a voltage is applied to an electrode, and become unstable once the voltage is turned off. In this case, the oxide exists in situ only.

As used herein, “electrochemically active molecule” refers to a molecule which can lose electrons to an electrode or accept electrons from an electrode when an appropriate voltage is applied to the electrode.

As used herein, “reducing agent” refers to any reagent that removes oxygen, contributes hydrogen, or contributes electrons. The reducing agent is oxidized in the reduction process. The relative strengths of reducing agents can be inferred from their standard electrode potentials. By convention, the standard electrode potentials are reduction potentials, or the tendency to be reduced. Thus, the strongest reducing agents will have large negative electrode potentials. (See e.g., Bard and Faulkner, Electrochemical Methods, Wiley, New York, 1980).

As used herein, “said reducing agent is not capable of being oxidized directly by said electrode” means that, although the difference between the standard potential of the reducing agent and the voltage applied to the electrode is large enough to oxidize the reducing agent, the speed of the oxidation is so slow it is negligible.

As used herein, “Good” buffers refer to the class of buffers introduced by Good, et. al. in 1966 (Good, N. E. et al., Biochemistry, 5:467 (1966)). They are zwitterionic buffers which contain secondary and tertiary amines as the positively charged groups, and sulfonic and carboxylic acids as the negatively charged groups. The exemplary Good buffers include N,N-Bis(2-hydroxyethyl)-2-aminoethanesulfonic acid (BES) (or N,N-Bis(2-hydroxyethyl)taurine), N,N-Bis(2-hydroxyethyl)glycine (BICINE), 3-(Cyclohexylamino)-1-propanesulfonic acid (CAPS), 4-(2-Hydroxyethyl)piperazine-1-propanesulfonic acid (HEPPS) (or N-(2-Hydroxyethyl)piperazine-N′-(3-propanesulfonic acid)), 4-(2-Hydroxyethyl)piperazine-1-ethanesulfonic acid (HEPES) (or N-(2-Hydroxyethyl)piperazine-N′-(2-ethanesulfonic acid)), 2-(N-Morpholino)ethanesulfonic acid (MES), or hemisodium salt or monohydrate thereof, 4-Morpholinepropanesulfonic acid (MOPS) (or 3-Morpholinopropanesulfonic acid), piperazine-1,4-bis(2-ethanesulfonic acid) (PIPES) (or piperazine-N,N′-bis(2-ethanesulfonic acid) or 1,4-piperazinediethanesulfonic acid), [(2-Hydroxy-1,1-bis[hydroxymethyl]ethyl)amino]-1-propanesulfonic acid (TAPS) (or N-[Tris(hydroxymethyl)methyl]-3-aminopropanesulfonic acid), 2-[2-Hydroxy-1,1-bis(hydroxymethyl)ethylamino]ethanesulfonic acid (TES) (or TES Free Acid) and N-[2-Hydroxy-1,1-bis(hydroxymethyl)ethyl]glycine (TRICINE) (or N-[Tris(hydroxymethyl)methyl]glycine) (see generally, the Sigma-Aldrich product catalog).

As used herein, “label” refers to any atom, molecule or moiety which can be used to provide a detectable signal.

As used herein, “antibody” refers to specific types of immunoglobulin, i.e., IgA, IgD, IgE, IgG, e.g., IgG₁, IgG₂, IgG₃, and IgG₄, and IgM. An antibody can exist in any suitable form and also encompass any suitable fragments or derivatives. Exemplary antibodies include a polyclonal antibody, a monoclonal antibody, a Fab fragment, a Fab′ fragment, a F(ab′)₂ fragment, a Fv fragment, a diabody, a single-chain antibody and a multi-specific antibody formed from antibody fragments.

As used herein, “nucleic acid” refers to any nucleic acid containing molecule including, but not limited to DNA, RNA or PNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, “plant” refers to any of various photosynthetic, eucaryotic multi-cellular organisms of the kingdom Plantae, characteristically producing embryos, containing chloroplasts, having cellulose cell walls and lacking locomotion.

As used herein, “animal” refers to a multi-cellular organism of the kingdom of Animalia, characterized by a capacity for locomotion, nonphotosynthetic metabolism, pronounced response to stimuli, restricted growth, and fixed bodily structure. Non-limiting examples of animals include birds such as chickens, vertebrates such fish and mammals such as mice, rats, rabbits, cats, dogs, pigs, cows, ox, sheep, goats, horses, monkeys and other non-human primates.

As used herein, “bacteria” and “bacterium” refer to all prokaryotic organisms, including those within all of the phyla in the Kingdom Procaryotae. The term encompass all microorganisms considered to be bacteria including Mycoplasma, Chlamydia, Actinomyces, Streptomyces, and Rickettsia. All forms of bacteria are included within this definition, including cocci, bacilli, spirochetes, spheroplasts, protoplasts, etc.

As used herein, “virus” refers to minute infectious agents, which with certain exceptions, are not observable by light microscopy, lack independent metabolism, and are able to replicate only within a living host cell. The individual particles (i.e., virions) consist of nucleic acid and a protein shell or coat. Some virions also have a lipid containing membrane. The term “virus” encompasses all types of viruses, including animal, plant, phage, and other viruses.

As used herein, “fungus” refers to a division of eucaryotic organisms that grow in irregular masses, without roots, stems, or leaves, and are devoid of chlorophyll or other pigments capable of photosynthesis. Each organism (thallus) is unicellular to filamentous, and possesses branched somatic structures (hyphae) surrounded by cell walls containing glucan or chitin or both, and containing true nuclei.

As used herein, “sample” refers to anything which may contain an analyte to be assayed using the present methods and/or devices. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). Biological tissues may be processed to obtain cell suspension samples. The sample may also be a mixture of cells prepared in vitro. The sample may also be a cultured cell suspension. In case of the biological samples, the sample may be crude samples or processed samples that are obtained after various processing or preparation on the original samples. For example, various cell separation methods, e.g., magnetically activated cell sorting) may be applied to separate or enrich target cells from a body fluid sample such as blood. Samples used for the present invention include such target-cell enriched cell preparation.

As used herein, a “liquid (fluid) sample” refers to a sample that naturally exists as a liquid or fluid, e.g., a biological fluid. A “liquid sample” also refers to a sample that naturally exists in a non-liquid status, e.g., solid or gas, but is prepared as a liquid, fluid, solution or suspension containing the solid or gas sample material. For example, a liquid sample can encompass a liquid, fluid, solution or suspension containing a biological tissue.

As used herein, “analyte” refers to any material that is to be analyzed. Such materials include, but are not limited to, ions, molecules, antigens, bacteria, compounds, viruses, cells, antibodies, and cell parts, etc.

As used herein, “specific binding” refers to the binding of one material to another in a manner dependent upon the presence of a particular molecular structure. For example, a receptor will selectively bind ligands that contain the chemical structures complementary to the ligand binding site(s). In contrast, “non-specific binding” refers to interactions that are arbitrary and not based on structural compatibilities of the molecules.

As used herein, “specific binding pair” refers to any substance, or class of substances, which has a specific binding affinity for the ligand to the exclusion of other substances. In one embodiment, the specific binding pair includes specific binding assay reagents which interact with the sample ligand or the binding capacity of the sample for the ligand in an immunochemical manner. For example, there will be an antigen-antibody or hapten-antibody relationship between reagents and/or the sample ligand or the binding capacity of the sample for the ligand. Additionally, it is well understood in the art that other binding interactions between the ligand and the binding partner serve as the basis of specific binding assays, including the binding interactions between hormones, vitamins, metabolites, and pharmacological agents, and their respective receptors and binding substances. (See e.g., Langan et al. eds., Ligand Assay, pp. 211 et seq., Masson Publishing U.S.A. Inc., New York, 1981).

As used herein, “plasma” refers to the fluid, noncellular portion of the blood, distinguished from the serum obtained after coagulation.

As used herein, “serum” refers to the fluid portion of the blood obtained after removal of the fibrin clot and blood cells, distinguished from the plasma in circulating blood.

As used herein, “fluid” refers to any composition that can flow. Fluids thus encompass compositions that are in the form of semi-solids, pastes, solutions, aqueous mixtures, gels, lotions, creams, and other such compositions.

As used herein the term “assessing” is intended to include quantitative and/or qualitative determination of an analyte present in the sample, and also of obtaining an index, ratio, percentage, visual or other value indicative of the level of the analyte in the sample. Assessment may be direct or indirect and the chemical species actually detected need not of course be the analyte itself but may for example be a derivative thereof or some further substance.

As used herein, “alkyl” encompasses straight or branched alkyl groups, including allyl groups that are optionally substituted with one or more substituents. For example, the alkyl group can be optionally substituted with hydroxy, halogen, aryl, alkoxy, acyl, or other substituents known in the art. One of more carbon atoms of the alkyl group can also be optionally replaced by one or more heteroatoms.

As used herein, “substitute” refers to the replacement of a hydrogen atom in a compound with a substituent group.

B. Electrochemistry Based Methods and Kits for Assaying an Analyte

In one aspect, the present invention is directed to a method for assaying an analyte, which method comprises: a) providing a reactant capable of binding and/or reacting with an analyte to be analyzed on an oxide electrode; b) contacting a sample suspected of containing said analyte with said reactant provided in step a) under suitable conditions to allow binding of said analyte, if present in said sample, to said reactant, wherein said reactant, said analyte, or additional reactant or additional analyte or analyte analog is covalently linked to an electrochemically active molecule in a reduced form, and said contacting brings said electrochemically active molecule into close proximity to said electrode to allow oxidation of said electrochemically active molecule by said electrode; c) allowing reduction of said oxidized electrochemically active molecule back to said reduced form by a reducing agent, wherein said reducing agent is not capable of being oxidized directly by said electrode, and said reduced electrochemically active molecule participates in said oxidation-reduction reactions of steps b) and c) repeatedly to generate an amplified electrochemical signal; and d) assessing said amplified electrochemical signal to determine presence and/or amount of said analyte in said sample.

In another aspect, the present invention is directed to a kit for assaying an analyte, which kit comprises: a) a reactant capable of binding and/or reacting with an analyte to be analyzed on an oxide electrode; b) an additional reactant, analyte, or analyte analog that is covalently linked to an electrochemically active molecule in a reduced form, wherein contacting of said analyte with said reactant on said electrode in the presence of said additional reactant, analyte, or analyte analog that is covalently linked to said electrochemically active molecule brings said electrochemically active molecule into close proximity to said electrode to allow oxidation of said electrochemically active molecule by said electrode; c) a reducing agent, wherein said reducing agent is not capable of being oxidized directly by said electrode, and said reducing agent reduces said oxidized electrochemically active molecule back to said reduced form to participate in repeated oxidation-reduction reactions to generate an amplified electrochemical signal; and d) means for assessing said amplified electrochemical signal to determine presence and/or amount of said analyte in said sample.

Preferably, the kit further comprises an instruction for using the kit to assay the analyte. Also preferbaly, the kit further comprises an oxide electrode that is capable of oxidzing the electrochemically active molecule but is not capable of oxidzing the reducing agent.

The present methods and kits can be used to assay any analyte such as a cell, a cellular organelle, a virus, a molecule and an aggregate or complex thereof. Exemplary cell includes an animal cell, a plant cell, a fungus cell, a bacterium cell, a recombinant cell and a cultured cell. Exemplary cellular organelle inlcudes a nuclei, a mitochondrion, a chloroplast, a ribosome, an ER, a Golgi apparatus, a lysosome, a proteasome, a secretory vesicle, a vacuole and a microsome. The molecule to be assayed can be an inorganic molecule, an organic molecule and a complex thereof. Exemplary organic molecule inlcudes an amino acid, a peptide, a protein, a nucleoside, a nucleotide, an oligonucleotide, a nucleic acid, a vitamin, a monosaccharide, an oligosaccharide, a carbohydrate, a lipid and a complex thereof. In a specific embodiment, the analyte to be assayed is a hormone, a cancer marker, a steroid, a sterol, a pharmaceutical compound, a metabolite of a pharmaceutical compound or a complex thereof.

The present methods and kits can be used to assay any suitable sample. For example, the present methods and kits can be used to assay an analyte in a mammalian sample, e.g., a sample derived from bovine, goat, sheep, equine, rabbit, guinea pig, murine, human, feline, monkey, dog and porcine. In another example, the present methods and kits can be used to assay an analyte in a clinical sample, e.g., serum, plasma, whole blood, sputum, cerebral spinal fluid, amniotic fluid, urine, gastrointestinal contents, hair, saliva, sweat, gum scrapings and tissue from biopsies. Preferably, the sample to be assayed is a human clinical sample. In still another example, the present methods and kits can be used to assay an analyte in a body fluid sample.

Any sutiable reactants can be used in the present methods and kits. Prefreably, the reactant binds and/or reacts specifically with the analyte. In one example, the reactant is a cell, a cellular organelle, a virus, a molecule and an aggregate or complex thereof. In another example, the reactant is an antibody. In still another example, the reactant is a nucleic acid.

The present methods and kits can be used in any suitable assay formats. In one example, the present methods and kits are used in a direct assay format wherein the analyte is covalently linked to an electrochemically active molecule and the contact between the reactant on the electrode with the analyte brings the electrochemically active molecule into close proximity to the electrode. In another example, the present methods and kits are used in a sandwich assay format wherein the reactant on the electrode, the analyte and a second reactant capable of binding and/or reacting with the analyte and covalently linked to an electrochemically active molecule forms a sandwich and brings the electrochemically active molecule into close proximity to the electrode. In still another example, the present methods and kits are used in a competition assay format wherein the analyte and an analyte or analyte analog with a covalently linked electrochemically active molecule competes for the binding with the reactant on the electrode and the binding of the analyte or analyte analog with the covalently linked electrochemically active molecule with the reactant brings the electrochemically active molecule into close proximity to the electrode. In yet another example, the present methods and kits are used in an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, chemiluminescence assay, lateral flow immunoassay, μ-capture assay, inhibition assay, energy transfer assay, avidity assay, turbidometric immunoassay or time resolved amplified cryptate emission (TRACE) assay.

Any sutiable electrochemically active molecules can be used in the present methods and kits. For example, the electrochemically active molecule can be a transition metal complex, e.g., a ferrocene, a metal porphyrin, a metal polypyridine, a metal poly-phenanthroline and a metal phthalocyanine. In one specific embodiment, the transition metal complex is a metal tris(2,2′-bipyridine) or one of its derivatives. In another specific embodiment, the transition metal complex is a ruthenium tris(2,2′-bipyridine) or one of its derivatives. Any suitable transition metal can be used. Exemplary transition metals inlcude cobalt, nickle, osmium, iron, rehnium, chromium and ruthenium.

The oxide electrode used in the present methods and kits can be formed in any suitable manner, e.g., formed in situ. Any suitable electrodes can be used in the present methods and kits. For example, the electrode can be a gold, platinum, silver, cobalt, nickel and carbon electrode. Preferably, the electrode is a metal oxide electrode. Either a single metal oxide or a combination of two or more metal oxides can be used. Exemplary metal oxides inlcude indium oxide, tin oxide, titanium oxide, zirconium oxide, tungsten oxide, zinc oxide and iron oxide. The metal oxide can be a pure metal oxide or a doped metal oxide, e.g., a tin-doped indium oxide or a florine-doped tin oxide.

Any sutiable reducing agents can be used in the present methods and kits. In one example, the reducing agent is soluble in an aqueous solution. In another example, the reducing agent is an organic redox molecule. Examplary organic redox molecules inlcude an organic acid, e.g., a carboxylic acid and oxalic acid, an organic base, e.g., an amine such as a primary, a secondary, or a tertiary amine, an organic ion, and an organic zwitterion. The organic redox molecule can also be an ionized organic acid, e.g., oxalate, or an ionized organic base, e.g., a protonated tripropyl amine. Any sutiable organic zwitterions can be used in the present methods and kits. For example, the organic zwitterion can comprise an organic base and an organic acid. In one specific embodiment, the organic base is an amine and the organic acid is a carboxylic acid. In another specific embodiment, the organic base is an amine and the organic acid is a sulfonic acid. In still another specific embodiment, the organic zwitterion is an amino acid, e.g., proline. In yet another specific embodiment, the organic zwitterion is a “Good” buffer, e.g., BES, BICINE, CAPS, HEPPS, HEPES, MES, MOPS, PIPES, TAPS, TES and TRICINE.

### C. EXEMPLARY EMBODIMENTS

In one specific embodiment, the present invention provides a system in which (1) an electrochemically active label is covalently attached to one molecule of the affinity binding pair; (2) the label undergoes fast electron transfer with an electrode where the affinity reaction takes place; (3) the label catalyzes electrochemical reaction of a chemical agent dissolved in solution; and (4) the electrode material is such that direct electrochemical reaction of the chemical agent is minimized. This system therefore offers high sensitivity because of amplified signal and low background.

For a sandwich immunoassay, the invention can be implemented with the following steps.

One of the antibody/antigen affinity pair (capture antibody) is immobilized on an electrode.

The electrode is contacted with a test sample. When the antigen is present, it binds to the immobilized capture antibody.

The electrode is then contacted with a second antibody to which an electrochemical label is covalently attached (labeled antibody). A tertiary complex of capture antibody/antigen/labeled antibody is thus formed.

Finally, the electrode is immersed in a solution containing a reducing agent. Chemically amplified electrochemical current of the label is measured, and correlated with the presence or amount of antigen in the test sample.

The embodiment is not limited to immunoassays, but applicable to other affinity-based assays as well, such as ligand-receptor, DNA-DNA, DNA-RNA, protein-DNA binding pairs. The analytes can be naturally occurring or synthetic chemical, biochemical or biological molecules, including drugs, peptides, proteins, ligands, receptors, sugars, vitamins, hormones, lipids, oligonucleotides, DNAs, RNAs, viruses, and cells. Assays can be either sandwich, competitive, or direct.

The electrochemical labels to be used herein, in general, have the following characteristics: (1) They exhibit fast electrochemical reaction with the electrode used in the detection; (2) Both oxidized and reduced sate of the molecule is stable; (3) They have functional groups that can be used to link to other molecules; (4) They are easy and inexpensive to synthesize and purify; and (5) When they are linked to other molecules, the affinity reaction is not affected in any significant detrimental way. Suitable labels are mostly transition metal complexes, such as ferrocenes, metal porphyrins, metal polypyridines, metal poly-phenanthrolines, and metal phthalocyanines. Metal polypyridines are attractive candidates as electrochemical labels because, besides the characteristics described above, their redox potential can be tuned in a wide range simply by using a different metal. For example, by using cobalt, osmium, iron and ruthenium, the redox potential of metal tris(2,2′-bipyridine) increases progressively from 0 V to 1.1 V vs. SCE. This variation in redox potential is desirable when one is looking for a good match in redox potential between a label and a reducing agent, as illustrated in Example 3.

To be suitable for chemically amplified electrochemical detection, the electrodes must be capable of fast electron exchange with the label, but negligible electrochemical reaction with the reducing agent. Many oxidized electrodes fulfill this requirement. They can be roughly classified into two groups, depending on how the electrodes get oxidized. The first group of electrodes is normally in the atomic state, but can be easily oxidized during electrochemical measurement. In other words, the oxidized electrodes are formed in situ. The oxidized state is not stable once the voltage is removed. These electrodes include gold, platinum, silver, cobalt, nickel, carbon, etc. The second group includes metal oxide electrodes, which are stable oxides. The materials include indium oxide, tin oxide, titanium oxide, zirconium oxide, tungsten oxide, etc. They can be either pure metal oxide, or doped, such as tin-doped indium oxide (ITO). Preparation of metal oxide electrodes of various shape and size is common knowledge to the skilled persons in this field. Preferred electrodes for this embodiment are metal oxides. Most preferred are doped and undoped indium oxide, tin oxide, and titanium oxide.

A reducing agent must be redox active, and its redox potential lower than that of the labeling molecule so as to be able to reduce the oxidized label. Also, its own electrochemical current must be kept at minimum to maximize detection sensitivity. In addition, the agent preferably haa adequate solubility in aqueous solution, and long shelf life. In general, organic redox molecules exhibit slow electrochemical reaction on most of the oxidized electrodes. The reason is not clear at present, but the common view is that the electrochemical reaction involves chemical bonding between the molecule and the metal surface. On the oxidized surface, such bonds can not form, thus slowing down the electron transfer reaction. Exemplary reducing agents include organic acid, organic base, organic ions, and organic zwitterions. These reducing agents may fall in into the categories of saturated alkyl, unsaturated alkyl, aromatic, or heterocyclic compounds. They may have substitute groups such as —OH, —F, —Cl, —Br, —I, —SH, etc. Organic acids include mono-carboxylic acid, di-carboxylic acid, and more. Preferred organic acids are di-carboxylic. Most preferred is oxalic acid. Organic base include mono-amines and poly-amines, and the amines may be primary, secondary, or tertiary. Preferred organic bases are tertiary amines. Most preferred is tripropyl amine. Furthermore, ionized forms of above organic acids and bases are also suitable as reducing agents for chemical amplification of electrochemical signal. Most preferred are oxalate and protonated tripropyl amine. Some organic zwitterions, such as amino acids and biological buffering molecules, contain both carboxylic acid and anine. They have also been found to be suitable reducing agent. Preferred organic zwitterions include proline, PIPES, and HEPES.

### D. EXAMPLES

### Example 1

### Oxalate-Amplified Electrochemical Current of Ruthenium tris-(bipyridine)

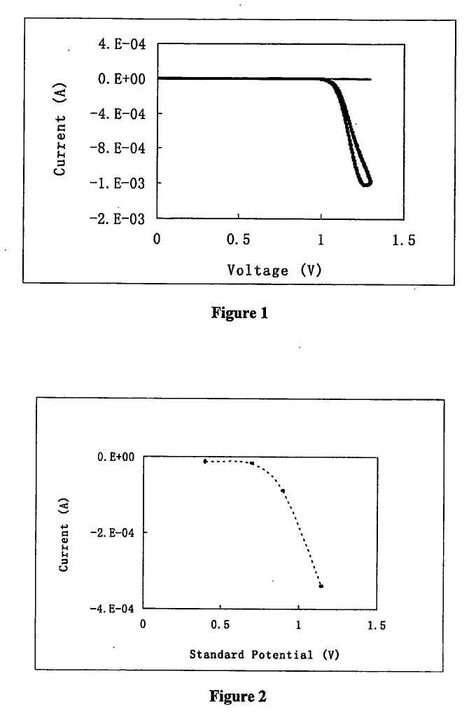

Ruthenium tris(2,2′-bipyridine) was purchased from Alfa Aesar, sodium oxalate from Avocado. A solution was prepared which contained 10 mM sodium oxalate, 0.5 mM ruthenium tris-(bipyridine), 0.1M sodium phosphate, pH 5.5. Electrochemical measurement was performed on a CHI 630A electrochemical analyzer. The working electrode was indium tin oxide film coated on glass slide with an area of 0.8 cm². A platinum foil was used as the counter electrode, and saturated calomel as reference. To perform the measurement, electrode voltage was scanned from 0 V to 1.3 V then back to 0 V at a rate of 100 mV/s. The current during the scan was recorded. The current was plotted against the voltage, as illustrated in FIG. 1. The thick line is for the solution containing ruthenium tris-(bipyridine), whereas the thin line is for the solution without the metal complex.

### Example 2

### Oxalate-Amplified Electrochemical Current of Various Metal Complexes

Ferrocene monocarboxylic acid was purchased from Alfa Aesar. Osmium and iron tris(2,2′-bipyridine) were synthesized according to the literature [please cite the reference].

Solutions were prepared which contained 10 mM sodium oxalate, 0.5 mM metal complex, 0.1M sodium phosphate, pH 5.5. Electrochemical measurement was performed as described in Example 1. The maximum current for each metal complex was plotted as a function of its standard potential, as illustrated in FIG. 2. Ruthenium tris(2,2′-bipyridine), which has the most oxidizing standard potential, produced the largest current.

### Example 3

### Electrochemical Current of Ruthenium tris(2,2′-bipyridine) Amplified by Various Reducing Agents

Reducing agents were purchased from the following vendors, and were used without further purification, PIPES (ICN), tripropyl amine (Alfa Aesar), HEPES (Avocado), proline (Alfa Aesar), tributyl amine (Shanghai United Chemicals, Shanghai, China), triehtyl amine (Shanghai United Chemicals, Shanghai, China).

Solutions were prepared which contained 10 mM reducing agent in 0.1M sodium phosphate. After electrochemical measurement of the reducing agent to get background current, ruthenium tris(2,2′-bipyridine) was added to a final concentration of 0.5 mM. The amplified current was measured in the same way as the background current. Amplification factor was obtained by first dividing the amplified current with the background for all the voltages, then choosing the maximum. The data in Table 1 below shows that oxalate had the highest amplification factor.

TABLE 1
Amplification factor of various reducing
agents (all pH 7.5 except noted)

| Reducing Agent | Amplification Factor |
|---|---|
| Sodium Oxalate (pH 5.5) | 2800 |
| Formic Acid | 1 |
| Triethyl Amine | <238 |
| Tripropyl Amine | 238 |
| Tributyl Amine | <238 |
| Proline | 580 |
| HEPES (pH 8.5) | 550 |
| PIPES (pH 8.5) | 175 |
| Gunosine | <<238 |

### Example 4

### Proline Amplified Current of Ruthenium Tris(2,2′-bipyridine) At Various Concentrations

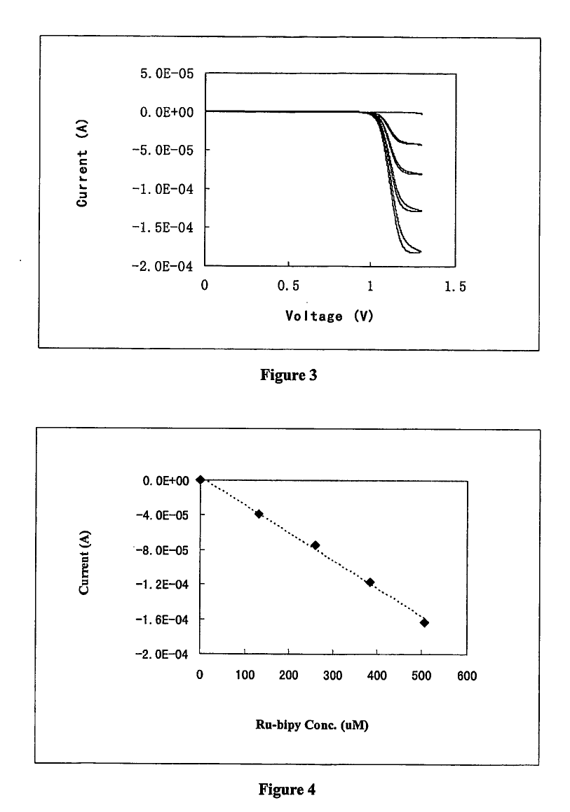

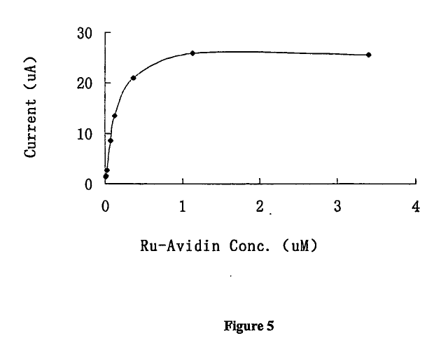

A solution containing 10 mM proline, 0.1M sodium phosphate, pH 7.5 was prepared. After electrochemical measurement of the reducing agent to get background current, ruthenium tris(2,2′-bipyridine) was added to a final concentration of 125 uM, 250 uM, 375 uM, and 500 uM. Proline amplified current for each ruthenium complex concentration was measured (FIG. 3). The current was found to be linear with the ruthenium concentration (FIG. 4).

### Example 5

### Biotin-Avidin Binding Detected by Chemically Amplified Electrochemistry

Biotin labeled bovine serum albumin (Biotin-BSA) was adsorbed onto indium-tin oxide electrode by immersing the electrode in a 1 mg/mL biotin-BSA solution for 1 hour at room temperature. After rinsing with a phosphate buffer, the electrode was immersed for 1 hour at room temperature in a solution of avidin labeled with ruthenium (4,4′-dicarboxyl)-bis(2,2′-bipyridine). The electrode was rinsed again with phosphate buffer. It was then placed into an electrochemical cell containing 10 mM sodium oxalate in 0.1M phosphate, pH 5.5. Electrochemical current was measured as described in Example 1. The current as a function of avidin concentration is illustrated in FIG. 5.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

What is claimed is:

1. A method for assaying an analyte, which method comprises:
a) providing a reactant capable of binding and/or reacting with an analyte to be analyzed on an oxide electrode;
b) contacting a sample suspected of containing said analyte with said reactant provided in step a) under suitable conditions to allow binding of said analyte, if present in said sample, to said reactant, wherein said reactant, said analyte, or additional reactant or additional analyte or analyte analog is covalently linked to an electrochemically active molecule in a reduced form, and said contacting brings said electrochemically active molecule into close proximity to said electrode to allow oxidation of said electrochemically active molecule by said electrode;
c) allowing reduction of said oxidized electrochemically active molecule back to said reduced form by a reducing agent, wherein said reducing agent is not capable of being oxidized directly by said electrode, and said reduced electrochemically active molecule participates in said oxidation-reduction reactions of steps b) and c) repeatedly to generate an amplified electrochemical signal; and
d) assessing said amplified electrochemical signal to determine presence and/or amount of said analyte in said sample.

2. The method of claim 1, wherein the analyte is selected from the group consisting of a cell, a cellular organelle, a virus, a molecule and an aggregate or complex thereof.

3. The method of claim 2, wherein the cell is selected from the group consisting of an animal cell, a plant cell, a fungus cell, a bacterium cell, a recombinant cell and a cultured cell.

4. The method of claim 2, wherein the cellular organelle is selected from the group consisting of a nuclei, a mitochondrion, a chloroplast, a ribosome, an ER, a Golgi apparatus, a lysosome, a proteasome, a secretory vesicle, a vacuole and a microsome.

5. The method of claim 2, wherein the molecule is selected from the group consisting of an inorganic molecule, an organic molecule and a complex thereof.

6. The method of claim 5, wherein the organic molecule is selected from the group consisting of an amino acid, a peptide, a protein, a nucleoside, a nucleotide, an oligonucleotide, a nucleic acid, a vitamin, a monosaccharide, an oligosaccharide, a carbohydrate, a lipid and a complex thereof.

7. The method of claim 1, wherein the analyte is selected from the group consisting of a hormone, a cancer marker, a steroid, a sterol, a pharmaceutical compound, a metabolite of a pharmaceutical compound and a complex thereof.

8. The method of claim 1, wherein the sample is a mammalian sample.

9. The method of claim 8, wherein the mammal is selected from the group consisting of bovine, goat, sheep, equine, rabbit, guinea pig, murine, human, feline, monkey, dog and porcine.

10. The method of claim 1, wherein the sample is a clinical sample.

11. The method of claim 10, wherein the clinical sample is selected from the group consisting of serum, plasma, whole blood, sputum, cerebral spinal fluid, amniotic fluid, urine, gastrointestinal contents, hair, saliva, sweat, gum scrapings and tissue from biopsies.

12. The method of claim 10, wherein the clinical sample is a human clinical sample.

13. The method of claim 1, wherein the sample is a body fluid sample.

14. The method of claim 1, wherein the reactant binds and/or reacts specifically with the analyte.

15. The method of claim 1, wherein the reactant is selected from the group consisting of a cell, a cellular organelle, a virus, a molecule and an aggregate or complex thereof.

16. The method of claim 1, wherein the reactant is an antibody.

17. The method of claim 1, wherein the reactant is a nucleic acid.

18. The method of claim 1, which is used in a direct assay format wherein the analyte is covalently linked to an electrochemically active molecule and the contact between the reactant on the electrode with the analyte brings the electrochemically active molecule into close proximity to the electrode.

19. The method of claim 1, which is used in a sandwich assay format wherein the reactant on the electrode, the analyte and a second reactant capable of binding and/or reacting with the analyte and covalently linked to an electrochemically active molecule forms a sandwich and brings the electrochemically active molecule into close proximity to the electrode.

20. The method of claim 1, which is used in a competition assay format wherein the analyte and an analyte or analyte analog with a covalently linked electrochemically active molecule competes for the binding with the reactant on the electrode and the binding of the analyte or analyte analog with the covalently linked electrochemically active molecule with the reactant brings the electrochemically active molecule into close proximity to the electrode.

21. The method of claim 1, wherein the electrochemically active molecule is a transition metal complex.

22. The method of claim 21, wherein the transition metal complex is selected from the group consisting of a ferrocene, a metal porphyrin, a metal polypyridine, a metal poly-phenanthroline and a metal phthalocyanine.

23. The method of claim 21, wherein the transition metal is selected from the group consisting of cobalt, nickle, osmium, iron, rehnium, chromium and ruthenium.

24. The method of claim 21, wherein the transition metal complex is a metal tris(2,2′-bipyridine) or one of its derivatives.

25. The method of claim 21, wherein the transition metal complex is ruthenium tris(2,2′-bipyridine) or one of its derivatives.

26. The method of claim 1, wherein the oxide electrode is native or formed in situ.

27. The method of claim 26, wherein the electrode is selected from the group consisting of gold, platinum, silver, cobalt, nickel and carbon electrode.

28. The method of claim 1, wherein the electrode is a metal oxide electrode.

29. The method of claim 28, wherein the electrode is a single metal oxide or a combination of two or more metal oxides.

30. The method of claim 28, wherein the metal oxide is selected from the group consisting of indium oxide, tin oxide, titanium oxide, zirconium oxide, tungsten oxide, zinc oxide and iron oxide.

31. The method of claim 28, wherein the metal oxide is a pure metal oxide or a doped metal oxide.

32. The method of claim 31, wherein the doped metal oxide is a tin-doped indium oxide.

33. The method of claim 31, wherein the doped metal oxide is a florine-doped tin oxide.

34. The method of claim 1, wherein the reducing agent is soluble in an aqueous solution.

35. The method of claim 1, wherein the reducing agent is an organic redox molecule.

36. The method of claim 35, wherein the organic redox molecule is selected from the group consisting of an organic acid, an organic base, an organic ion and an organic zwitterion.

37. The method of claim 36, wherein the organic acid is a carboxylic acid.

38. The method of claim 36, wherein the organic acid is oxalic acid.

39. The method of claim 36, wherein the organic base is an amine.

40. The method of claim 36, wherein the organic base is a primary, a secondary, or a tertiary amine.

41. The method of claim 36, wherein the organic base is tripropyl amine.

42. The method of claim 35, wherein the organic redox molecule is an ionized organic acid or an ionized organic base.

43. The method of claim 42, wherein the ionized organic acid is oxalate.

44. The method of claim 42, wherein the ionized organic base is protonated tripropyl amine.

45. The method of claim 36, wherein the organic zwitterion comprises an organic base and an organic acid.

46. The method of claim 45, wherein the organic base is an amine and the organic acid is a carboxylic acid.

47. The method of claim 45, wherein the organic base is an amine and the organic acid is a sulfonic acid.

48. The method of claim 36, wherein the organic zwitterion is an amino acid.

49. The method of claim 48, wherein the amino acid is proline.

50. The method of claim 36, wherein the organic zwitterion is a “Good” buffer.

51. The method of claim 50, wherein the “Good” buffer is selected from the group consisting of BES, BICINE, CAPS, HEPPS, HEPES, MES, MOPS, PIPES, TAPS, TES and TRICINE.

52. The method of claim 1, which is used in an assay format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, chemiluminescence assay, lateral flow immunoassay, μ-capture assay, inhibition assay, energy transfer assay, avidity assay, turbidometric immunoassay and time resolved amplified cryptate emission (TRACE) assay.

53. A kit for assaying an analyte, which kit comprises:
a) a reactant capable of binding and/or reacting with an analyte to be analyzed on an oxide electrode;
b) an additional reactant, analyte, or analyte analog that is covalently linked to an electrochemically active molecule in a reduced form, wherein contacting of said analyte with said reactant on said electrode in the presence of said additional reactant, analyte, or analyte analog that is covalently linked to said electrochemically active molecule brings said electrochemically active molecule into close proximity to said electrode to allow oxidation of said electrochemically active molecule by said electrode;
c) a reducing agent, wherein said reducing agent is not capable of being oxidized directly by said electrode, and said reducing agent reduces said oxidized electrochemically active molecule back to said reduced form to participate in repeated oxidation-reduction reactions to generate an amplified electrochemical signal; and
d) means for assessing said amplified electrochemical signal to determine presence and/or amount of said analyte in said sample.

54. The kit of claim 53, which further comprises an instruction for using the kit to assay the analyte.

55. The kit of claim 53, which further comprises an oxide electrode that is capable of oxidzing the electrochemically active molecule but is not capable of oxidzing the reducing agent.
